(19) United States
(12) Patent Application Publication
(10) Pub. No.: US 2023/0067042 A1
(43) Pub. Date: Mar. 2, 2023

(54) Medical Leg Placer or Placing Device

(71) Applicants: Jiheon Kang, Evanston, IL (US); Seungheon Kang, Evanston, IL (US)

(72) Inventors: Jiheon Kang, Evanston, IL (US); Seungheon Kang, Evanston, IL (US)

(21) Appl. No.: 17/464,956

(22) Filed: Sep. 2, 2021

(51) Int. Cl. A61F 5/01

(52) U.S. Cl. CPC A61F 5/0127

(57) ABSTRACT

Provided is a medical leg placing device to place a users injured leg, the medical leg placing device comprising: a first placer extending in an oblique direction with respect to the ground to place a calf of the leg; and a second placer coupled to one surface of the first placer and extended to place a foot of the leg.

This invention enables an injured leg to place stably without interruption both in a horizontal direction to the ground by crossing two supports coupled to the other surface of the first placer and in an oblique direction by unfolding a pair of supports.

It can be used comfortably for a patient with an injured leg sitting on a chair by adjusting the heights of the supports of a first placer.

### TECHNICAL FIELD

The present disclosure relates to a medical leg placing device.

### BACKGROUND ART

In general, patients who injured their legs are often moved using medical boots, crutches or wheelchairs.

The medical improvement has been quite evolved in which to protect the injured legs of the patients, or improve the inconvenience of the movement.

However, when a patient who injured the legs is sitting on a chair, it is difficult to find any device for their convenience. When a patient is sitting on a chair, the injured leg can not be extended toward the floor as in the general leg. At this time, the patient requires a separate chair or device to place his injured leg.

Thus, there is a problem that other person or the patient himself should find this separate chair or device. In addition, this separate chair or device can not be easily carried, so the patient always experiences inconvenience that this chair or device should be procured in the place where the patient is sitting.

### DESCRIPTION OF EMBODIMENTS

### Technical Problem

Provided is a medical leg placing device that is easy to be carried and allows a patient sitting in a chair to comfortably place his or her injured leg.

### Solution to Problem

According to an aspect of the present disclosure, a medical leg placing device is provided to place a user's injured leg, the medical leg placing device may comprise a first placer extending in an oblique direction with respect to a ground to place a calf of the leg; and a second placer coupled to one surface of the first placer and extended to place a foot of the leg.

The medical leg placing device may further comprise a first support coupled to the other surface of the first placer and fixing the first placer to the ground.

One end of the first support may be hinged to the first placer.

One end of the second placer may be hinged to the first placer.

The first placer may further comprise a second support installed to sandwich the calf therebetween.

The second support may be spaced apart from one end of the first placer by a first distance, the other end of the second placer 12 may be spaced apart from the first placer by a second distance, and the first distance may be longer than the second distance.

The medical leg placing device may further comprise a coupling device coupled to the first placer and fixing the first placer to a chair on which the user sits.

Other aspects, features, and advantages than those described above will be clear from the accompanying drawings, the claims, and the description of embodiments below.

### Advantageous Effects of Disclosure

According to an embodiment, a user does not need to find a separate chair or tool to put his/her leg on, since the user can easily place his/her leg when he or she sits in a chair.

The medical leg placing device according to the embodiments of the present invention is provided to be portable, foldable and thus can be easily used by a user anytime, anywhere.

Since the medical leg placing device according to the embodiments of the present invention can stably support the injured leg of a patient, it is possible to relieve discomfort caused by the user's injured leg.

This invention enables an injured leg to place stably without interruption both in a horizontal direction to the ground by crossing two supports coupled to the other surface of the first placer and in an oblique direction by unfolding a pair of supports.

It can be used comfortably for a patient with an injured leg sitting on a chair by adjusting the heights of the supports of a first placer.

### BRIEF DESCRIPTION OF DRAWINGS

FIG. 1 is a perspective view of a medical leg placing device according to an embodiment of the present disclosure.

FIG. 2 is a front view of a medical leg placing device according to an embodiment of the present disclosure.

FIG. 3 is a side view of a medical leg placing device according to an embodiment of the present disclosure.

FIG. 4 is a top view of a medical leg placing device according to other embodiment of the present disclosure.

FIG. 5 is a rear view of a medical leg placing device according to another embodiment of the present disclosure.

FIG. 6 is a drawing shown a portion of a medical leg placing device according to another embodiment of the present disclosure.

FIG. 7 is a side view of a medical leg placing device according to another embodiment of the present disclosure.

### MODE OF DISCLOSURE

Hereinbelow, various embodiments and/or modes are shown and described with reference to the drawings. For the purpose of explanation, a variety of specific details are described to promote understanding one or more modes. However, it will be recognized by one of ordinary skill in the art that such mode(s) can be implemented without these specific details. The descriptions below and the attached drawings show in detail particular example modes of one or more modes. However, these modes are examples, and some of various methods based on the principles of various modes may be used, and the descriptions below are intended to include all these modes and equivalents.

In addition, various modes and features will be presented by systems that may include devices, components, and/or modules. It will be appreciated and understood that various systems may include additional devices, components, and/or modules and/or may not include all of devices, components, and modules described with reference to the drawings.

It may not be interpreted that the terms “embodiment”, “example”, “mode”, “illustration”, etc. used herein specify that a stated mode or design is better or more advantageous than other modes or designs. It will be further understood that the terms “comprises” and/or “comprising” specify the presence of relevant features and/or components, but do not preclude the presence or addition of one or more other features, components, and/or groups thereof.

While terms including ordinal numbers such as “first,” “second,” etc., may be used to describe various components, such components must not be limited to the above terms. The above terms are used only to distinguish one component from another. For example, a first component could be termed a second component, and, similarly, a second component could be termed a first component without departing from the scope of the present disclosure. The term “and/or” includes combinations of a plurality of associated listed items or one of the associated listed items.

Unless otherwise defined, all terms (including technical and scientific terms) used herein have the same meaning as commonly understood by one of ordinary skill in the art to which the present disclosure belongs. It will be understood that terms, such as those defined in commonly used dictionaries, should be interpreted as having a meaning that is consistent with their meaning in the context of the relevant art, and will not be interpreted in an idealized or overly formal sense unless expressly so defined herein.

Some components are omitted, enlarged, or reduced in the drawings to explain the functions of each component, but it will be understood that the details shown in the drawings do not limit the technical features and scope of the present disclosure.

In the descriptions below, a technical feature or a component will be described with reference to a plurality of drawings together.

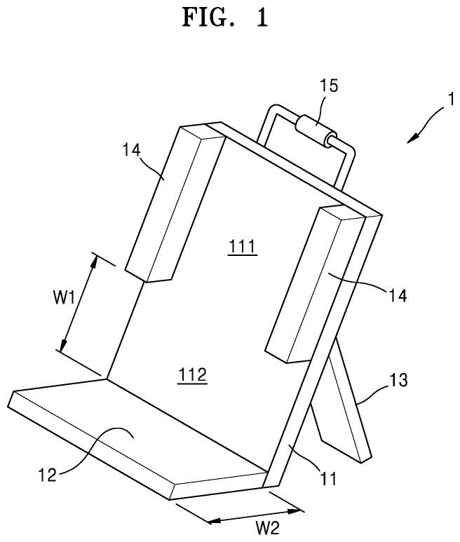

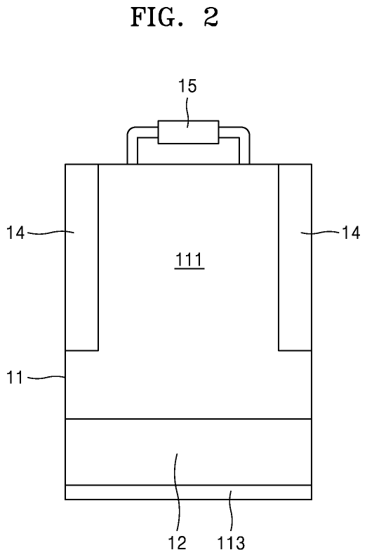

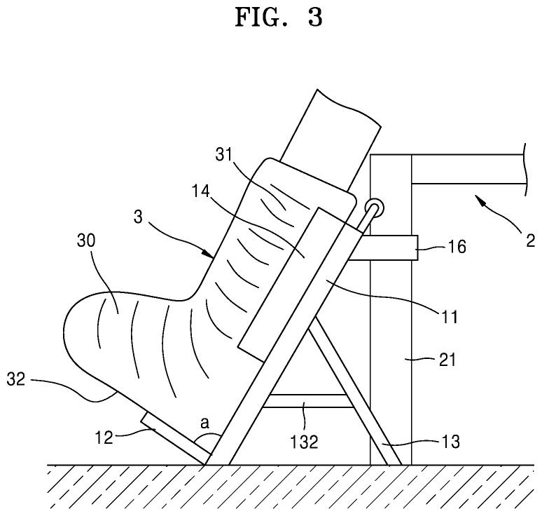

FIG. 1 is a perspective view of a medical leg placing device according to an embodiment of the present disclosure. FIG. 2 is a front view of a medical leg placing device according to an embodiment of the present disclosure. FIG. 3 is a side view of a medical leg placing device according to an embodiment of the present disclosure.

Referring to FIGS. 1-3, a medical leg placing device 1 according to an embodiment of the present invention may be provided to place a user's injured leg 3. According to one embodiment, the medical leg placing device 1 is provided so that the user can put the user's injured leg 3 when he/she sits on a chair 2, and thus It is possible to reduce an effort of finding a separate chair or a tool to put the injured leg 3 on when the user sits on the chair 2. According to one embodiment, the user may be in a state of wearing a medical boot 30 for protecting the injured leg 3, and thus the medical leg placing device 1 may be a device for placing the leg wearing the boot.

An embodiment of the medical leg placing device 1 comprises a first placer 11 extending in an oblique direction with respect to the ground to place a calf 31 of the leg 3, and a second placer 12 coupled to the first placer 11 and extended to place a foot 32 of the leg 3.

The first placer 11 may be provided on a flat plate, and comprises a first region 111 mainly placing the calf 31 of the user's leg 3, and a second region 112 being adjacent to the first region 111 in a direction of the ground from the first region 111. The second placer 12 may be coupled to the first placer 11 mainly in the second region 112.

One end of the second placer 12 may be hinged to the first placer 11, and accordingly, the second placer 12 may be rotated by an angle a about the first placer 11 as shown in FIG. 3. The second placer 12 may be in close contact with the second region 112 of one surface of the first placer 11 when it is folded with the first placer 11, and one end of the second placer 12 may be closely fixed to a third region 113 of one surface of the first placer 11 when it is unfolded. The angle a may be approximately 90 degrees, but is not necessarily limited thereto and may be set within a range of 60 degrees to 120 degrees. Although not shown in the drawings, the second placer 12 may be hinged to the first placer 11 so that the angle a is variable. In this case, the hinge may be a device for arbitrarily setting an angle.

In this way, since the second placer 12 is provided to form a predetermined angle with respect to the first placer 11 when it is unfolded, the user puts the calf 31 on the first placer 11 and the foot 32 on the second placer 12 when using the medical leg placing device 1. And when the user does not use the medical leg placing device 1, the second placer 12 is folded to be in close contact with the first placer 11, so that the user can simply carry the medical leg placing device 1.

A carrier 15 that can be held by hand may be further installed on the first placer 11 so that the user can carry it more conveniently.

This medical leg placing device 1 can be used when a user sits on a chair, and may include a coupling device 16 that can be coupled to the chair 2 as shown in FIG. 3. According to one embodiment, the coupling device 16 is coupled to the other surface of the first placer 11 and may be a device for fixing the first placer 11 to a leg 21 of the chair 2, for example, Velcro may be used.

Selectively, the medical leg placing device 1 may further include a first support 13 for fixing the first placer 11 to the ground.

One end of the first support 13 is hinged to the other surface of the first placer 11, and accordingly, the first support 13 may be rotated at a predetermined angle from the first placer 11. One end of the first support 13 supports the first placer 11 and the other end of the first support 13 supports the ground when it is unfolded, so that the first placer 11 can be stably supported in a state with a certain inclination with respect to the ground. A connecting rod 132 may be further installed between the first support 13 and the first placer 11 to maintain an angle between the first placer 11 and the first support 13. The first support 13 may be in close contact with the other surface of the first placer 11 in the folded state, so that the user can easily carry the medical leg placing device 1.

According to other embodiment, a pair of the first support 13 is placed at both ends of the first placer 11 in its width direction, the user uses the first placer 11 in tilting state by unfolding only one first support 13.

According to one embodiment, the medical leg placing device 1 may further include a second support 14 installed to sandwich the calf 31 therebetween. The second support 14 may be coupled to one surface of the first placer 11, and more specifically, may be coupled to the first region 111 of the first placer 11. According to an embodiment, the second support 14 is provided as a pair, and may be installed at both ends of the first region 111 in the width direction. Due to the second support 14, when the user puts the calf 31 on the first placer 11, the user can obtain more stability. The second support 14 may be made of a material having a cushioning function, for example, a sponge material may be used. The distance between the pair of second supports 14 can be adjusted according to the user's selection, so that it can correspond to the user's leg thickness.

The second support 14 may be installed at a position that does not interfere with the second placer 12 when the second placer 12 is folded toward the first placer 11. To this end, the second support 14 is spaced apart from one end of the first placer 11 by a first distance W1, the other end of the second placer 12 is separated from the first placer 11 by a second distance W2, and the first distance W1 may be longer than the second distance W2. Therefore, when the user carries the medical leg placing device 1, the second placer 12 can be folded so as to be in close contact with the first placer 11 without being interfered with by the second support 14, and portability can be simplified.

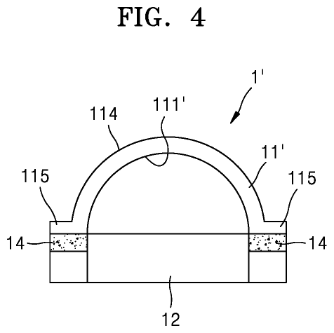

FIG. 4 is a top view of a medical leg placing device according to other embodiment of the present disclosure.

According to the embodiment shown in FIG. 4, the first placer 11′ of the medical leg placing device 1′ may include a portion 114 having a curvature. The curved portion 114 is provided to be rounded to fit the user's leg, and accordingly, the first region 111′ placing the user's leg, particularly the calf, has a curved surface. Since the user's leg is stably supported by the curved portion 114, the user can find more stability.

According to another embodiment, selectively the first placer 11′ may comprise planar portions 115 adjacent to the curved portion 114, the planar portions 115 can be located at both ends of the curved portion 114 in the width direction.

Selectively, the second support 14 may be coupled to the planar portion 115, and the second placer 12 may be coupled to the planar portion 115. Accordingly, when the second placer 12 is folded, the second placer 12 can be in close contact with the planar portion 115, and the user can simply carry the medical leg placing device 1′.

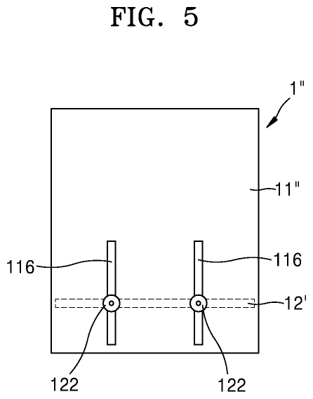

FIG. 5 is a rear view of a medical leg placing device according to another embodiment of the present disclosure.

According to the embodiment shown in FIG. 5, slits 116 extending in a direction perpendicular to the ground may be formed in the first placer 11″. According to another embodiment, the slits 116 may be formed in the second region of the first placer 11″. A position adjusting device 122 coupled to the second placer 12 may be installed in the slits 116, and the second placer 12 may change its position in the extending direction of the slit 116 by the position adjusting device 122. Therefore, it is possible to adjust the second placer 12 to a higher position to fit a short user. Although not shown in the drawings, the second placer 12 may be hinge-coupled to the position adjusting device 122, and accordingly, even when the position is adjusted in the extending direction of the slit 116, It is possible to be fold toward the first placer 11.

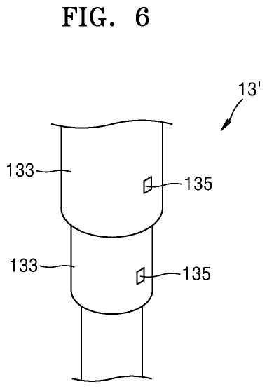

Meanwhile, FIG. 6 shows another embodiment of the first support 13′. According to another embodiment, the first support 13′ may be provided so as to be stretchable in the longitudinal direction thereof. For example, the first support 13′ may include a plurality of pipes 133 inserted to overlap each other. The pipes 133 may be fixed while being pulled out from each other by the fixing button 135. By the first support 13′ as described above, the user can adjust the angle or height of the first support 11 with respect to the ground.

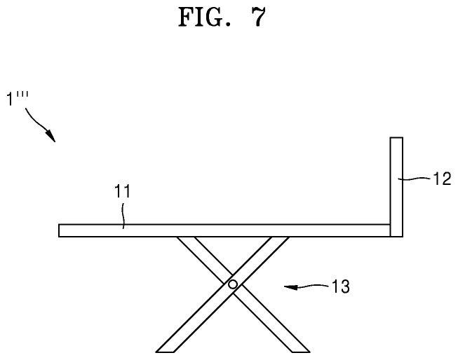

FIG. 7 shows a medical leg placing device 1′″ according to another embodiment.

According to the embodiment shown in FIG. 7, the angle of the first placer 11 can be set in a direction substantially horizontal to the ground. In this case, the first support 13 preferably supports the first placer more firmly so that the first placer 11 can be maintained horizontally. For example, the first support 13 is installed in an X-shape, so that the first placer 11 is stably supported when the first placer 11 is in substantially horizontal state. In another embodiment, the first supports 13 may be installed one by one at the corners of the first placer 11 so that the medical leg placing device 1′″ maintains a table shape. At this time, each of the first supports 13 may be hinged so as not to interfere with each other when folded.

Of course, all the embodiments of the present specification described above are not limited to the illustrated embodiments, and may be applied in combination with each other.

It is apparent that combinations of the embodiments described above may be implemented. In other words, features of one of the embodiments may also be applied to other embodiments.

While embodiments have been described with reference to particular embodiments and drawings, various changes and modifications may be made in the above descriptions by those of ordinary skill in the art. For example, even when the techniques described above are performed in a different order than described above, and/or the components such as systems, structure, devices, circuits, etc. described above are coupled to or combined with each other in different manners than described above or substituted or replaced with other components or equivalents, proper results may be obtained. Therefore, other implements, other embodiments, and equivalents to the scope of the claims are included in the scope of the claims described below.

What is claimed is:

1. A medical leg placing device provided to place a user's injured leg, the medical leg placing device comprising:
a first placer extending in an oblique direction with respect to the ground to place a calf of the leg; and
a second placer coupled to one surface of the first placer and extended to place a foot of the leg.

2. The medical leg placing device of claim 1, further comprising a pair of the first support coupled to the other surface of the first placer and fixing the first placer to the ground.

3. The medical leg placing device of claim 2, wherein one end of the first support is hinged to the first placer.

4. The medical leg placing device of claim 1, wherein one end of the second placer is hinged to the first placer.

5. The medical leg placing device of claim 1, wherein the first placer further comprises a second support installed to sandwich the calf therebetween.

6. The medical leg placing device of claim 5,
wherein the second support is spaced apart from one end of the first placer by a first distance,
wherein the other end of the second placer 12 is spaced apart from the first placer by a second distance, and
wherein the first distance is longer than the second distance.

7. The medical leg placing device of claim 1, further comprising a coupling device coupled to the first placer and fixing the first placer to a chair on which the user sits.
